(19) United States
(12) Patent Application Publication
(10) Pub. No.: US 2005/0251063 A1
(43) Pub. Date: Nov. 10, 2005

(54) Safety device for sampling tissue

(76) Inventor: Raghuveer Basude, Fremont, CA (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/841,906

(22) Filed: May 7, 2004

(51) Int. Cl. A61B 10/00; A61B 17/32

(52) U.S. Cl. 600/564; 600/567

(57) ABSTRACT

An apparatus for sampling tissue includes a tissue engaging member for engaging and retaining a sample of a tissue structure, and a cutter surrounding the tissue engaging member. The tissue engaging member and cutter are both operative to translate longitudinally with respect to one another, and a mechanism prevents the cutter from moving distally, the mechanism being released and the cutter freed to move distally when the tissue engaging member is in a predetermined position, preferably distal of the cutter. Thereby, out of sequence operation, e.g., premature cutting of the tissue before the tissue engaging member is in position, is prevented.

### BACKGROUND OF THE INVENTION

1. Field of Invention

The invention relates to the field of medical instruments, and more particularly, to a device for sampling tissue.

2. Description of Related Art

Tissue coring devices, e.g., punches, as well as other devices for sampling tissue, such as for biopsies, are used in many surgical applications. For example, in order to perform proximal anastomosis of a bypass graft on to the aorta, it is essential to perform an aortotomy. In such a procedure, a punch is passed through an opening created in the aorta by the punch or a separate instrument. Typically, the punch employs a device to retain the tissue to be cut by the coring knife of the punch such that, following the cut, the excised tissue is retained by the punch. A clean cylindrical aortotomy of the right size is essential to the success of the procedure. Moreover, the aortotomy should be free of tissue damage and embolic events. An embolic event can occur if the donut, i.e., the excised tissue, is lost in the body. The donut can then become an embolus that can travel in the bloodstream until it reaches a constricted region where it blocks blood flow.

Such a scenario can occur when the coring knife of the punch is used out-of-sequence. For example, where the coring knife is used to core or sample the tissue prior to deployment of a tissue retention device, the retention device can then deployed after the complete coring, pushing the cut donut into the blood stream. The out-of-sequence scenario can be caused by the surgeon's unfamiliarity with the device or procedure or a simple mental lapse, for example. Furthermore, out-of-sequence use of the punch can result in damage to the instrument and/or to the surrounding tissue.

Other problems with conventional devices occur where the coring knife is exposed and is damaged during use. A damaged coring knife is undesirable as it produces unreliable aortotomies. In addition, an exposed coring knife can damage the sealing device that may be used in conjunction with the punch. When used in aortotomy, a sealing device maintains a hermetic seal with the aorta after the aortotomy is formed, preventing excessive blood loss. It is known to use a cap to protect the coring knife when it is not being used, as well as during insertion of the punch into the handle. The cap is small, however, and can be dropped easily, as well, potentially damaging the coring knife.

Another weakness of the prior art is that it is possible with conventional devices to damage the aorta or other organ due to “back-walling,” which occurs where the coring knife strikes the tissue behind the tissue which is desired to be cut. Accordingly, there is a need for a device for sampling or coring tissue that addresses the above and other issues.

### BRIEF SUMMARY OF THE INVENTION

The present invention addresses the shortcomings of the prior art by providing an improved device for sampling or coring tissue. The device is suitable for use with essentially any type of tissue, such as tissue from the aorta, intestines and veins. Generally, the device is suitable for any biopsy or for the cutting and retrieval of tissue from any tissue structure. The device has a robust design that improves safety by positively securing the cut tissue or donut, while also providing a means to prevent out-of-sequence operation. Furthermore, the device can provide positive sensory feedback, e.g., auditory, visual, and/or tactile, regarding the configuration of the device. In one embodiment, the device has a recessed cutter or coring knife to prevent incidental damage and to eliminate the need for an additional cap.

According to the present invention, an apparatus for sampling tissue includes a tissue engaging for engaging and retaining a sample of a tissue structure, and a cutter surrounding the tissue engaging member. The tissue engaging member and cutter are both operative to translate longitudinally with respect to one another, and a mechanism prevents the cutter from moving distally, the mechanism being released and the cutter freed to move distally when the tissue engaging member is in a predetermined position, preferably distal of the cutter. Thereby, out of sequence operation, e.g., premature cutting of the tissue before the tissue engaging member is in position, is prevented.

Alternately or additionally, distal movement of the tissue engaging member to a first predetermined position initiates distal movement of the cutter to a second predetermined position, proximal of the tissue engaging member.

### BRIEF DESCRIPTION OF THE DRAWINGS

These and other features, advantages and benefits will be made apparent through the following descriptions and accompanying figures, where like reference numerals refer to the same features across the various drawings.

FIGS. 1(a)-(g) illustrate a sequence of use of a tissue sampling device according to the present invention;

FIG. 2 illustrates a deployment sequence of a cutter and retention member of a tissue sampling device according to the present invention;

FIG. 3(a) illustrates a schematic view of a tissue sampling device in a reset position according to the present invention;

FIG. 3(b) illustrates a schematic view of a tissue sampling device with its retention member deployed according to the present invention;

FIG. 3(c) illustrates a schematic view of a tissue sampling device with its retention member and cutter deployed according to the present invention;

FIG. 4(a) illustrates a profile view of a tissue sampling device with its retention member and cutter deployed according to the present invention; and

FIG. 4(b) illustrates a cross-sectional view of the tissue sampling device of FIG. 4(a) according to the present invention.

FIGS. 5(a)-5(d) illustrate the sequence of use of a tissue sampling device according to a second embodiment of the present invention.

### DETAILED DESCRIPTION OF THE INVENTION

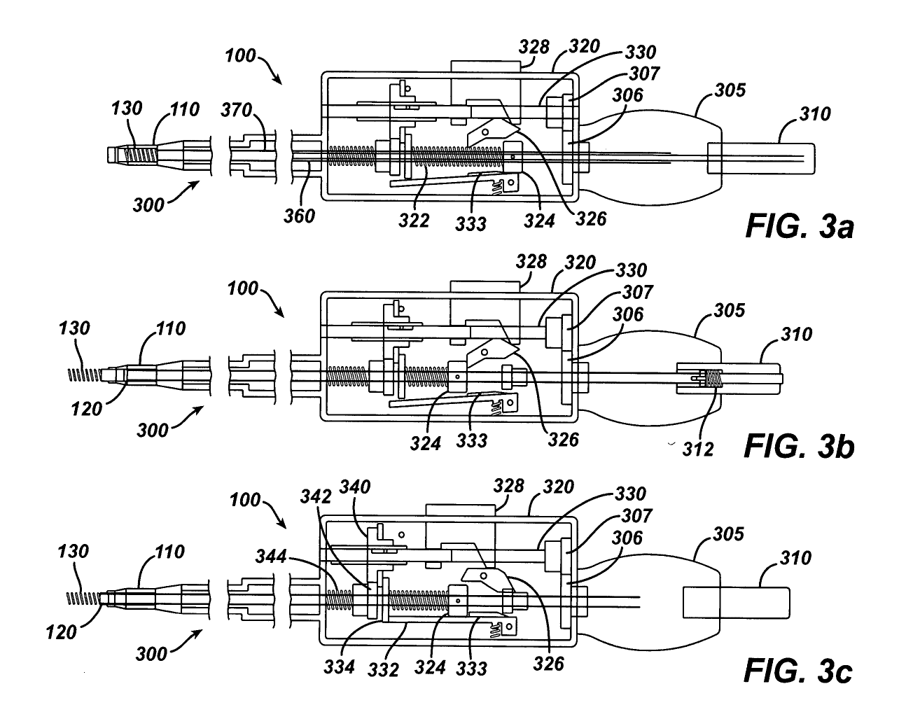

FIG. 3(a) illustrates a schematic view of a tissue-sampling device in a reset position according to one embodiment of the present invention. In the reset position of the device 100, the retention member 130 and cutter 120 are disposed within the outer sleeve 110 of the body 300. The device includes various actuating mechanisms, including a plunger 310 that, together with a control assembly 320, controls the translation of the retention member 130. The control assembly 320, shown schematically, preferably includes mechanisms for controlling the translation and rotation of the retention member 130 and cutter 120. Specifically, a spring 322 biases a block 324 rearward or proximally. The block 324 is linked to a shaft 360 on which the retention member 130 is carried. The plunger 310 includes a push-button spring lock mechanism 312, shown in FIG. 3(b), and is connected to the shaft 360.

In operation, the plunger 310 is depressed and moves the block 324, shaft 360 and retention member 130 distally against the force of the compressed spring 322. When the block 324 moves a predetermined distance in a distal direction, the block 324 forces a distal end of a lever 326 to move clockwise until the block 324 passes the lever 326, at which time a proximal face of the block 324 opposes a distal portion of the lever 326, preventing the block 324 from retracting proximally. The lever 326 is preferably pivotally mounted and spring-biased in the counterclockwise direction.

With the lever 326 opposed to block 324, the retention member 130 protrudes from the body 300, locked against proximal movement. Significant further movement in the distal direction is resisted by spring 322, which is preferably configured to be nearly at its fully compressed position when block 324 is distal to lever 326.

Since rotation of the block 324 is not precluded by contact with the lever 326, rotational movement of the shaft 360 and retention member 130 is allowed. In particular, rotational movement of the plunger 310 results in rotational movement of the shaft 360 and retention member 130. The retention member 130 can thus be rotated to engage the tissue to be cored. Once the surgeon visually observes that the retention member 130 has engaged the tissue, the surgeon advances knob 328 distally to deploy the cutter 120. Distal movement of the knob 328 advances the stop key 341, gears 340 and 342, and flange 334 distally against the urging of spring 344. Flange 334 is operatively connected to the cutter 120, and therefore the cutter is protruded from the body 300. Flange 334 is advanced until it is secured against proximal retraction by spring-biased lever 332. Lever 332 is prevented from moving into an engaged position by projection 333 before the distal extension of block 324. Accordingly, the cutter cannot be latched in a deployed position until the tissue retention member 130 is in a range of distal positions, i.e., in advance of the cutter 120. Once the cutter is deployed, the surgeon rotates a knob 305 that causes gears 306 and 307 to rotate. The gears 306 and 307 may have a 3:1 ratio, for example. Rotation of the gear 307 causes rotation of shaft 330.

As the block 328 moves distally, it contacts a proximal portion of the lever 326, causing the distal portion of the lever 326 to rotate clockwise to a position where the distal portion of the lever 326 does not contact the block 324, as shown in FIG. 3(c). Free from the restraint of lever 326, spring 322 urges block 324 and retention member 130 proximally. Therefore, the tissue captured by the retention member would be drawn into the body 300 once severed by the cutter 120. Additionally, as block 328 rides along the top portion of lever 326, a lower portion of lever 326 engages any of one or more slots 327 disposed on shaft 360, thereby preventing any rotational movement of shaft 360 and retention member 130.

Gears 340, 342 are positioned distally within control assembly 320 to control the movement of cutter 120. Gear 340 is slidably mounted onto shaft 330 such that as knob 305 is rotated, gear 340 rotates. Gear 342 is slideably disposed on shaft 360, and is engaged with gear 340. Gear 342 in turn is connected directly to flange 334, which is connected to the cutter 120 through a shaft 370. Shaft 370 may be mounted coaxially outside the shaft 360. The proximal end of gear 340 is attached to a key 341, which is disposed about shaft 330. At least a portion of key 341 is disposed between gear 342 and flange 334. As gear 340 moves distally due to the rotation of knob 305, key 341 ensures that gear 342 and flange 334 also move distally. Therefore, by turning knob 305, the surgeon both advances and rotates the cutter 120 to core the tissue engaged by the retention member 130.

As is shown in FIG. 3(a) and described, supra, lever 332 is initially biased against a lower portion of flange 334. With lever 326 in the position shown in FIG. 3(c), only the tissue itself prevents the retention member 130, the shaft 360 and block 324 from retracting proximally into the body 300 under the urging of spring 322. Therefore, when the cutter 120 completes the coring operation, the retention member 130, the shaft 360 and block 324 all retract automatically, capturing the tissue donut 195 within the body 300. Further, block 324 contacts projection 333 provided on lever 332, causing the lever 332 to pivot downward and out of engagement with the proximal side of flange 334. Subsequently, flange 334 and cutter 120 retract into the body under the urging of spring 344. The proximal movement of the cutter 120, flange 334, gears 340, 342, and key 341 may be limited by a fixed stop 343 provided within control assembly 320.

The return to the reset position proximally within body 300 provides a visual confirmation to the surgeon that the donut 195 has been captured and the coring is complete. The block 328 may protrude outside of the control assembly 320 so that the surgeon can visually ascertain the movement and position of the block 328 and, consequently, the cutter 120. That is, if the block 328 is positioned at a distal location, the cutter 120 is deployed, and if the block 328 is positioned at a proximal location, the cutter 120 is withdrawn. The distal portion of the device 100 may be disassembled and sterilized for reuse, if desired.

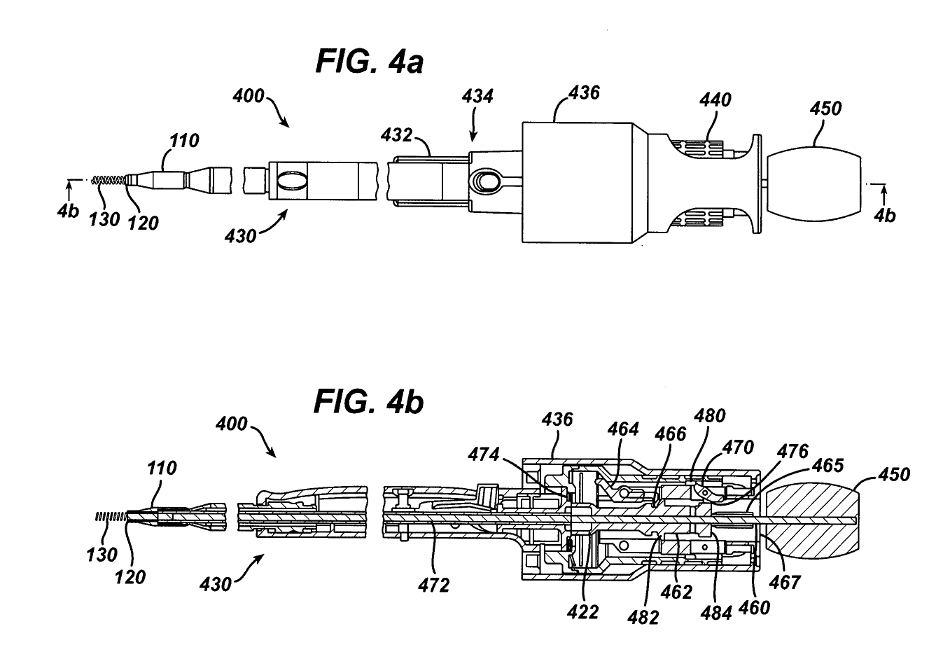

FIG. 4(a) illustrates a profile view of a tissue-sampling device with its retention member 130 and cutter deployed according to a second embodiment of the present invention. FIG. 4(b) illustrates a longitudinal cross-section of FIG. 4(a). The device 400 includes a body 430 having a distal portion 432 that is removable from a control assembly 436 via a handle release 434. Actuating mechanisms include a perforated or slotted wheel 440 that translates axially and rotates, to engage and rotate, respectively, the cutter 120. Knob 450 translates axially and rotates to control translation and rotation, respectively, of the retention member 130.

In a reset position, the knob 450 is positioned proximally from the control assembly 436, preferably further than shown. In particular, a shaft 460 is linked to the knob 450, detent structure 462, and retention member 130. When the knob 450 is pushed toward the control assembly 436, i.e., moved distally, against the force of a spring 422, a pivoting lever 464, which is spring biased in the clockwise direction, engages in a detent 466 of the detent structure 462. This locks the retention member 130 in the deployed position to prevent further axial movement. The engagement of the lever 464 in the detent 466 does not prevent rotation of the retention member 130 in response to rotation of the knob 450. One or more gear teeth or fins 465 are provided about the shaft 460. A corresponding cutout in the proximal end wall 467 of the control assembly 436 allows the shaft 460 and the retention member 130 to be withdrawn proximally and locked against rotation. The cutouts may also extend distally from the end wall 467 to resist rotation of the shaft 460 for a longer range of axial motion.

Perforated wheel 440 is shown in a distal position. In this position, gears 474 engage to allow the perforated wheel 440 to rotate the shaft 472 and ultimately the cutter 120. Additionally, when pressed distally against a proximal bias, in this case a coil spring, the shaft 472 is pressed distally to expose the cutter 120. Pivoting lever 470 is spring biased clockwise. In the reset position, lever 470 will not hold the perforated wheel 440 forward if it were advanced. However, when shaft 460 is engaged with latch 464 in a deployed position, proximal protrusion 484 constrains the movement of lever 470, preventing full rotation. Thereafter, when perforated wheel 440 is pressed distally, lever 470 engages a circumferential ridge 480 in the interior of perforated wheel 440. Once engaged, lever 470 prevents proximal movement of the perforated wheel 440, while allowing it to rotate. The shaft 472 may be coaxially outside the shaft 460 that carries the retention member 130.

With the retention member 130 deployed, the surgeon pierces and engages the tissue with the retention member 130. The surgeon may rotate the knob 450 to cause the coil-shaped retention member 130 to securely engage the tissue. If the retention member 130 comprises a barb, hook or other structure that does not require rotation to engage the tissue, the device 400 need not have that capability. Moreover, as mentioned, the entire device 400 can be rotated manually by the surgeon to rotate the retention member 130.

The distal movement of the knob 450 to extend the retention member 130 causes a raised structure 476 associated with the shaft 460 to contact the lever 470. Thus, lever 470 to pivots counterclockwise to the position shown in FIG. 4(b), where it disengages from the perforated wheel 440. This permits the surgeon to manually move the perforated wheel 440 distally to extend the cutter 120 from the body 430 of the device 400. Therefore, the cutter 120 cannot be deployed until after the retention member 130 is deployed. When the perforated wheel 440 is moved distally, a portion of the perforated wheel 440 contacts a portion of the lever 464, such as the upper portion which extends radially outward and distally, to move it counterclockwise, causing the lever 464 to disengage from the detent 466 in the detent structure 462. As a result, the retention member 130 is urged back proximally by the spring 422. However, the retention member 130 with the engaged tissue cannot move proximally under the spring bias because of the resiliency of the tissue. Subsequently, the surgeon manipulates the cutter 120, e.g., via the perforated wheel 440, to core and sample the tissue. The perforated wheel 440 is linked via gears 474 to the shaft 472 that controls the cutter 120.

As the tissue is being cored, the resiliency in the tissue allows limited proximal movement of the retention member influenced under spring 422. In a preferred embodiment, once so moved, fins 465 will engage slots 467 to prevent rotation of the shaft 460. Once the tissue is cored, the retention member 130 retracts proximally into the cutter 120 and body 430, and fins 465 pass through the corresponding cutout in the end wall 467. The shaft 460 and consequently the knob 450 are also retracted proximally, providing a visual confirmation to the surgeon that the donut 195 has been captured and the coring is complete. A protrusion 482 on the shaft 460 urges the lever 470 counterclockwise as it passes, freeing the perforated wheel 440 to retract distally, thereby allowing the cutter 120 to retract as well.

Thus, distal movement of the knob 450 results in locking of the retention member 130 in the deployed position and unlocking of the cutter 120. Subsequent distal movement of the perforated wheel 440 results in unlocking the retention member 130. Additional locking mechanisms may be provided as desired, e.g., to maintain the cutter 120 locked in the non-deployed position while the retention member 130 is locked in the deployed position.

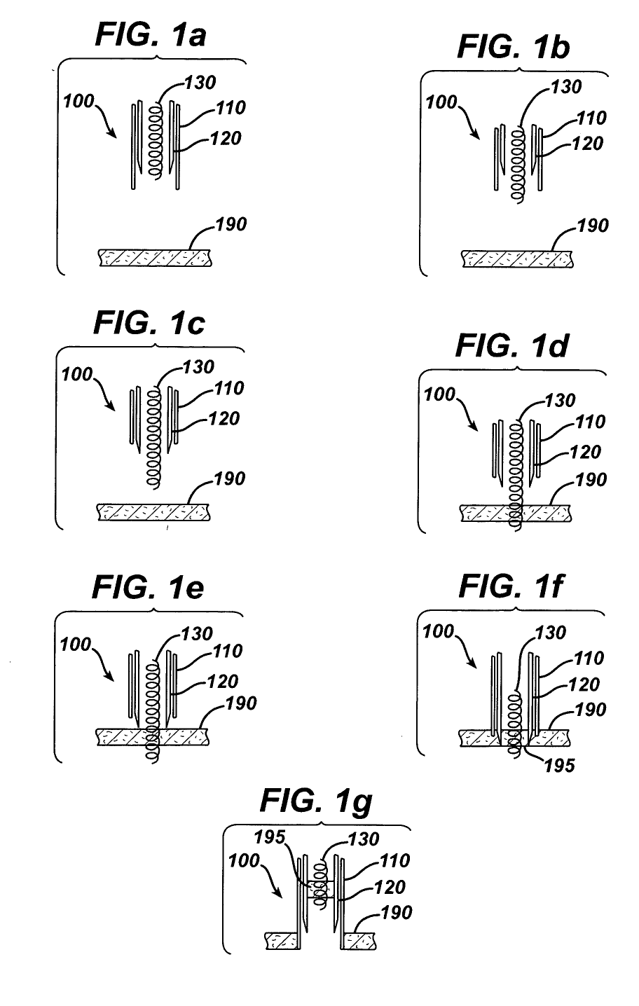

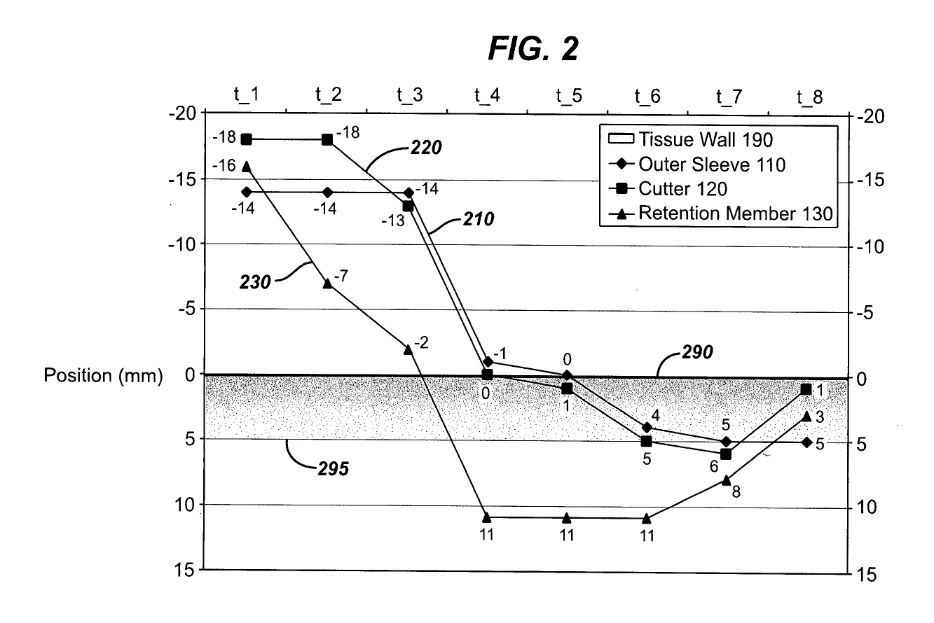

Referring now to FIGS. 1(a)-1(g) and the graph of FIG. 2, shown is a sequence of operation for a cutter according to the present invention. Each FIG. 1(a)-1(f) corresponds approximately to each instant in time t_1 through t_6, respectively, while FIG. 1(g) corresponds to instant t_8. In the graph of FIG. 2, the horizontal axis illustrates time, which is not necessarily continuous, and the vertical axis illustrates a relative position of the cutter 120, retention member 130 and outer sleeve 110 of the body with respect to the tissue wall 190. In the present example, the units on the vertical axis may be millimeters (mm). A position less than zero on the vertical axis indicates that the outer sleeve 110, the cutter 120, or the retention member 130 is outside the surface, or adventita 290, of the tissue wall 190, whose position is represented by the line 290. The inner surface of the tissue wall 190, or intima 295, presumes a wall thickness of 5 mm, solely for the purposes of illustration. The position of the outer sleeve 110 is represented by line 210, while the position of the retention member 130 is represented by line 230, and the position of the cutter 120 is represented by line 220. The reset configuration of the device in FIG. 1(a) is shown at time t_1 in FIG. 2. In this illustrative example, the cutter 120 is recessed 4 mm inside the end of the outer sleeve 110, while the retention member 130 is recessed 2 mm inside the end of the outer sleeve 110. The tissue 190 is 14 mm beyond the end of the outer sleeve 110.

The configuration shown in FIG. 1(b) corresponds with time t_2 in FIG. 2. Here, the retention member 130 protrudes or extends beyond the end of the outer sleeve 110, e.g., at a position 7 mm past the outer sleeve 110. The cutter 120 has not moved during the time between t_1 and t_2 when the retention member 130 initially protrudes past the end of the outer sleeve 110. However, following time t_2, the cutter 120 begins to move distally until it protrudes to its fullest extent, e.g., 1 mm past the outer sleeve 110, at time t_3, as shown in FIG. 1(c). The retention member 130 also continues to move distally between t_2 and t_3 until it protrudes fully, e.g., 12 mm past the outer sleeve 110.

At time t_3, the surgeon may advance the device 100 toward the tissue 190 until the tip of the retention member 130 pierces the tissue, FIG. 1(d). The coil-like retention member 130 may also be rotated at this time to further engage and draw the tissue toward cutter 120. If the retention member 130 is not mounted for rotation with respect to the body of the device, the retention member 130 can be rotated by having the surgeon manually rotated the entire device. If the retention member 130 is mounted for rotation with respect to the body of the device, the retention member 130 can be rotated by rotating or otherwise moving an actuating mechanism, as described supra. Optionally, the retention member may comprise a barb, hook, scoop or other structure that does not require rotation to engage the tissue.

At time t_3, when the retention member 130 protrudes fully, it may be locked to prevent further axial movement, while permitting the retention member 130 to rotate. Additionally, the cutter 120 may be locked to prevent both translation and rotation with respect to the retention member and/or the outer sleeve 110. Between time t_3 and t_4, the retention member 130 is rotated further to draw the tissue closer to the cutter 120. At time t_4, the tissue 190 contacts the cutter 120, FIG. 1(e), and rotation of the cutter 120 may be allowed, while the lock against translation is maintained. In particular, the surgeon rotates the cutter 120, and simultaneously advances it, either manually or through an actuating mechanism, until it cores the tissue 190, FIG. 1(f). The retention member 130 secures the resulting tissue core, or donut 195, to ensure it is not lost. In addition, a rotational lock on the retention member 130 may be engaged at this time, while any lock against translation has already been or is now disengaged to allow the retention member 130 to start to withdraw proximally, back into the outer sleeve, between time t_4 and t_5.

The retention member 130 may withdraw gradually by movement of an appropriate actuating mechanism. Alternately, the actuating mechanism may have a spring mechanism that exerts a force on the retention member 130 to cause the retention member 130 to snap back when the tissue is cored. The retention member 130 is withdrawn at least into the interior of the cutter 120 and eventually into the interior of the device body.

At time t_6, the intima of the tissue donut 195 is even with the cutter 120 and thus is fully cored as is represented by the intersection of lines 290 and 210. Withdrawal of the retention member 130 and the donut 195 continues through time t_6 up until time t_7 while the cutter 120 remains deployed. At time t_7, when the retention member 130 is fully withdrawn, withdrawal of the cutter 120 begins. At time t_8, the cutter 120 is also fully withdrawn into the outer sleeve 110, FIG. 1(g). If the retention member 130 retracts under the force of a spring mechanism when the donut 195 is free from the surrounding tissue 190, the time between t_6 and t_7 is essentially zero.

Preferably, when using the device, the retention member 130 should be at a position distal to the cutting knife 120 and fully deployed prior to the exposure of the cutting knife 120 beyond the outer sleeve 110. This ensures that the retention member 130 is fully deployed into the tissue 190 before the tissue 190 contacts the cutter 120. This configuration is depicted in FIG. 1(e), where the cutter 120 meets the tissue only after the retention member 130 has pierced and at least a portion of the retention member 130 tissue has passed into the tissue. At this point, a proximal spring bias may be applied to the retention member 130, and additionally, the retention member 130 may be prevented from rotating with respect to the tissue 190. Thus, only the cutter 120 is allowed to rotate, thereby coring the tissue as seen in FIG. 1(f). When the full wall thickness of the aorta or other tissue 190 is cut, the retention member 130 retracts inside the lumen of the cutter 120 along with the captured donut 195. At this time, the cutter 120 may be retracted so that both the cutter 120 and the retention member 130 retract well within the atraumatic outer lumen or sleeve 110 of the device body at time t8, as in the reset position of FIG. 1(a). Thus, the device 100 is reset.

Therefore, according to the present invention, the surgeon cannot core or even cut the tissue 190 prior to deploying the retention device 130, thereby preventing use of the device 100 out-of-sequence.

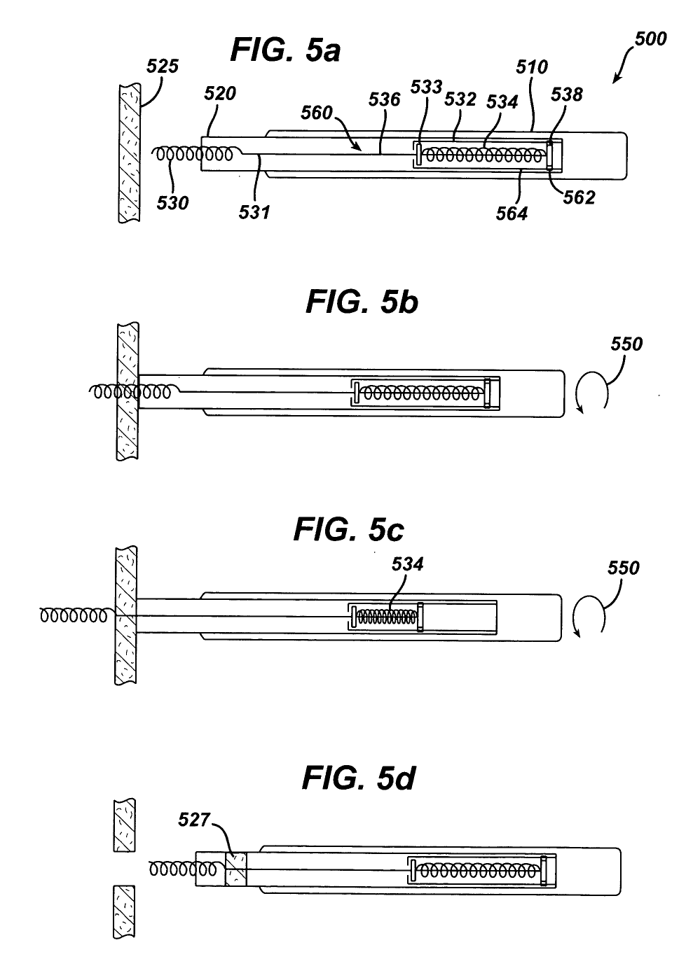

Referring now to FIGS. 5(a)-5(d), illustrated is a sequence of use of an example tissue-sampling device with a fixed cutter according to another embodiment of the present invention. A schematic view of the device 500 includes a body 510, a cutter 520, a retention member 530, and an actuating mechanism 560. The actuating mechanism 560 includes a chamber 532 in which a spring 534 is provided. The chamber is closed at its distal end, in this case by a fixed washer 533. Spring 534 extends between the fixed washer 533 and a movable plate 538 at the proximal end of the chamber 532. Plate 538 is fixed to the retention device 530 via shaft 536.

Further, plate 538 may be provided with one or more tabs 562, which project into one or more slots 564 provided along the length of the chamber 532. As a result, the plate 538 and the retention member 530 are constrained to rotate with the chamber 532. Tabs 562 and/or slots 564 may also extend to the exterior of the body 510, to provide a visual indication to the surgeon of the retention member position. Alternately or additionally, the body 510 may be mad transparent in whole or in part in order to make the tabs 562 visible to the surgeon.

The retention member 530 protrudes distally beyond the cutter 520. When the retention member 530 is a coil or other design that requires rotation, the surgeon rotates the chamber 532, or the body 510 if fixed to the chamber 532, to engage the tissue 525. Arrow 550 illustrates an example direction of rotation. Rotation continues until the retention member 530 engages the tissue and the cutter 520 begins to contact the tissue 525 (FIG. 5b). Furthermore, the actuating mechanism 530 may be actuated so that the retention member 530 is fed further distally while the spring 534 is compressed (FIG. 5c).

Once the cutter 520 contacts the tissue 525, the cutter can be rotated to core or otherwise cut the tissue. The tissue 525 is urged proximally against the cutter 520 by the force of the spring 534 that acts on the retention member 530 (FIG. 5c). When the tissue 525 has been cored, the retention member 530 with the tissue donut 527 secured thereon is free to snap back proximally under the force of the spring 534 (FIG. 5d). The retention member 530 also retracts, at least partly, in the cutter 520.

A lever, knob, or other equivalent means may also be provided for positioning and optionally, locking, the retention member 530 in the fully protruding position of FIG. 5c, where it protrudes past the cutter 530 to engage the tissue 525. Once the tissue 525 is engaged, the retention member 530 can be unlocked, at which time the tissue 525 is urged proximally so that the cutter 520 contacts the tissue 525, as shown in FIG. 5b. A proximal user-operable portion may be provided for rotating the cutter 520 as well to allow the cutter 520 to cut the tissue 525.

When the retention member 530 is a barb or other element that does not require rotational movement to engage the tissue 525, the retention member 530 can be mounted translation only with respect to the body 510. A lever, knob, or other equivalent means may again be provided for locking or otherwise positioning the retention member 530 in the position of FIG. 5c. Once the tissue is engaged, the retention member is unlocked and urged proximally so that the tissue 525 is urged against the cutter 520, as shown in FIG. 5c. Again, a proximal user-operable portion may be provided for rotating the cutter 520 to allow the cutter 520 to cut the tissue 525.

Advantageously, in the approach of FIGS. 5a-5d, the device 500 is simplified in construction since the cutter 520 can be fixed against translation relative to the body 510. It is also possible for the cutter to be fixed against rotation with respect to the body 510. In this case, the cutter 520 can be rotated by rotating the entire body 510. At least part of the retention member 530 always protrudes distally past the cutter 520. A protective cap may be employed when the device 500 is not in use, notwithstanding the disadvantages of a cap as described, supra.

Specific construction details that are not shown are believed to be within the purview of those of ordinary skill in the art. Moreover, note that any portion or all of the deployment sequence of the device, such as movement of the cutter 120 or retention member 130, can be automated using appropriate sensors and motorized actuating mechanisms.

The present invention has been described herein with reference to certain preferred embodiments. These embodiments are offered as illustrative, and not limiting, of the scope of the invention. Certain modifications or alterations may be apparent to those skilled in the art without departing from the scope of the invention, which is defined by the appended claims.

1. An apparatus for sampling tissue comprising:
a tissue engaging member disposed at a distal end of the apparatus, the tissue engaging member being operative to engage and retain a sample of a tissue structure;
a cutter disposed surrounding the tissue engaging member, wherein the tissue engaging member and cutter are both operative to translate longitudinally with respect to one another; and
a latch operative to hold the cutter distally, the latch operative to engage only when the tissue engaging member is in a predetermined range of positions.

2. The apparatus according to claim 1, further comprising a first actuator operative to translate the tissue engaging member.

3. The apparatus according to claim 2, wherein the first actuator is operative to rotate the tissue engaging member.

4. The apparatus according to claim 2, wherein the first actuator is positioned at a proximal end of the apparatus.

5. The apparatus according to claim 1, further comprising a second actuator operative to rotate the cutter.

6. The apparatus according to claim 0.5, wherein the second actuator is operative to translate the cutter.

7. The apparatus according to claim 5, wherein the second actuator is positioned at a proximal end of the apparatus.

8. The apparatus according to claim 1, further comprising an outer sleeve surrounding the cutter and tissue engaging member when the cutter and tissue engaging members are disposed in their respective proximal positions.

9. The apparatus according to claim 8, wherein the outer sleeve is tapered from a first diameter at the first end to a second diameter greater than the first diameter at a point proximal of the first end.

10. The apparatus according to claim 1, further comprising means for holding the tissue engaging member at the predetermined position.

11. The apparatus according to claim 11, wherein the means for holding the tissue engaging member at the predetermined position is released by a predetermined distal movement of the cutter.

12. The apparatus according to claim 1, wherein either or both of the cutter and the tissue engaging member are biased to translate proximally of the first end.

13. The apparatus according to claim 1, wherein the predetermined range of positions of the tissue engaging member comprises a range of positions beginning at a distal deployed position.

14. The apparatus according to claim 13, wherein the predetermined range of positions of the tissue engaging member extends in a distal direction from the distal deployed position.

15. An apparatus for sampling tissue, the apparatus comprising:
a body having a cutter at a first end; and
a tissue engaging member having its distal end positioned distally of the cutter, the tissue engaging member mounted to translate only distally of the cutter.

16. The apparatus according to claim 15, wherein the cutter is fixed to the body.

17. The apparatus according to claim 15, wherein the tissue engaging member is mounted to rotate unitarily with the body.

18. The apparatus according to claim 15, wherein the position of the tissue engaging member is indicated along the body of the apparatus.

19. An apparatus for sampling tissue comprising:
a tissue engaging member disposed at a first end of the apparatus and biased to move proximally of the first end, the tissue engaging member being operative to engage and retain a sample of a tissue structure; and
a cutter disposed surrounding the tissue engaging member and biased to move proximally of the first end, wherein the tissue engaging member and cutter are both operative to translate longitudinally with respect to one another;
wherein distal movement of the tissue engaging member to a first predetermined position permits the cutter to move to a second predetermined position proximal of the tissue engaging member.

20. The apparatus according to claim 19, further comprising a first lever operative to oppose the proximal movement of the tissue engaging member after the tissue engaging member has advanced to the first predetermined distal position.

21. The apparatus according to claim 20, wherein the release of the first lever by the cutter advancing to the second predetermined distal position allows the tissue engaging member to move proximally under its bias.

22. The apparatus according to claim 19, further comprising a second lever operative to oppose the proximal movement of the cutter once the cutter has advanced to the second predetermined distal position.

23. The apparatus according to claim 22, wherein the release of the second lever by the tissue engaging member moving to a proximal reset position allows the cutter to move proximally under its bias.

24. An apparatus for sampling tissue, comprising:
a body;
a cutter mounted for translation with respect to the body;
a retention member mounted for translation with respect to the body; and
a first portion linked to the retention member for translating the retention member from a non-deployed position to a deployed position;
a second portion linked to the cutter for translating the cutter from a non-deployed position to a deployed position;
a first latching mechanism responsive to distal translation of the first portion for latching the retention member; and
a second latching mechanism responsive to the distal translation of the first portion for latching the cutter.

25. The apparatus of claim 24, wherein the second latching mechanism is responsive to proximal translation of the second portion for unlatching the cutter.

26. The apparatus of claim 24, wherein the second latching mechanism is responsive to distal translation of the second portion for unlatching the retention member.

27. The apparatus of claim 24, wherein the retention member is carried within the cutter.

28. The apparatus of claim 24, wherein the cutter is carried within the body.

29. The apparatus of claim 24, wherein the first and second portions are manually operable.

30. An apparatus for sampling tissue, comprising:
a body;
a cutter mounted for translation with respect to the body;
a retention member mounted for translation with respect to the body; and
at least one actuating mechanism, carried at least partly within the elongated body, for controlling movement of the cutter and the retention member;
wherein the at least one actuating mechanism prevents deployment of the cutter out of sequence before the retention member.

31. The apparatus of claim 29, wherein the at least one actuating mechanism is manually operable.
